(19) United States
(12) Patent Application Publication
(10) Pub. No.: US 2016/0045433 A1
(43) Pub. Date: Feb. 18, 2016

(54) Children's Medication Delivery Device

(71) Applicant: Debbie Whitney, Motgomery, AL (US)

(72) Inventor: Debbie Whitney, Motgomery, AL (US)

(21) Appl. No.: 14/458,359

(22) Filed: Aug. 13, 2014

(51) Int. Cl. A61K 9/00

(52) U.S. Cl. CPC A61K 9/0056

(57) ABSTRACT

A children's medication delivery device for delivering liquid medication to a young child in a form that is palatable and attractive to facilitate administering medication to the child. The device includes a shell comprising a gummy candy composition. The shell defines an interior space within the shell. A medication is positioned in the interior space of the shell wherein the medication is enclosed by and contained in the shell.

### BACKGROUND OF THE DISCLOSURE

### Field of the Disclosure

The disclosure relates to medication delivery devices and more particularly pertains to a new medication delivery device for delivering liquid medication to a young child in a form that is palatable and attractive to facilitate administering medication to the child.

### SUMMARY OF THE DISCLOSURE

An embodiment of the disclosure meets the needs presented above by generally comprising a shell comprising a gummy candy composition. The shell defines an interior space within the shell. A medication is positioned in the interior space of the shell wherein the medication is enclosed by and contained in the shell.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top front side perspective view of a children's medication delivery device according to an embodiment of the disclosure.

FIG. 2 is a back view of an embodiment of the disclosure.

FIG. 3 is a side view of an embodiment of the disclosure.

FIG. 4 is a front view of an embodiment of the disclosure.

FIG. 5 is a cross-sectional view of an embodiment of the disclosure taken along line 5-5 of FIG. 4.

### DESCRIPTION OF THE PREFERRED EMBODIMENT

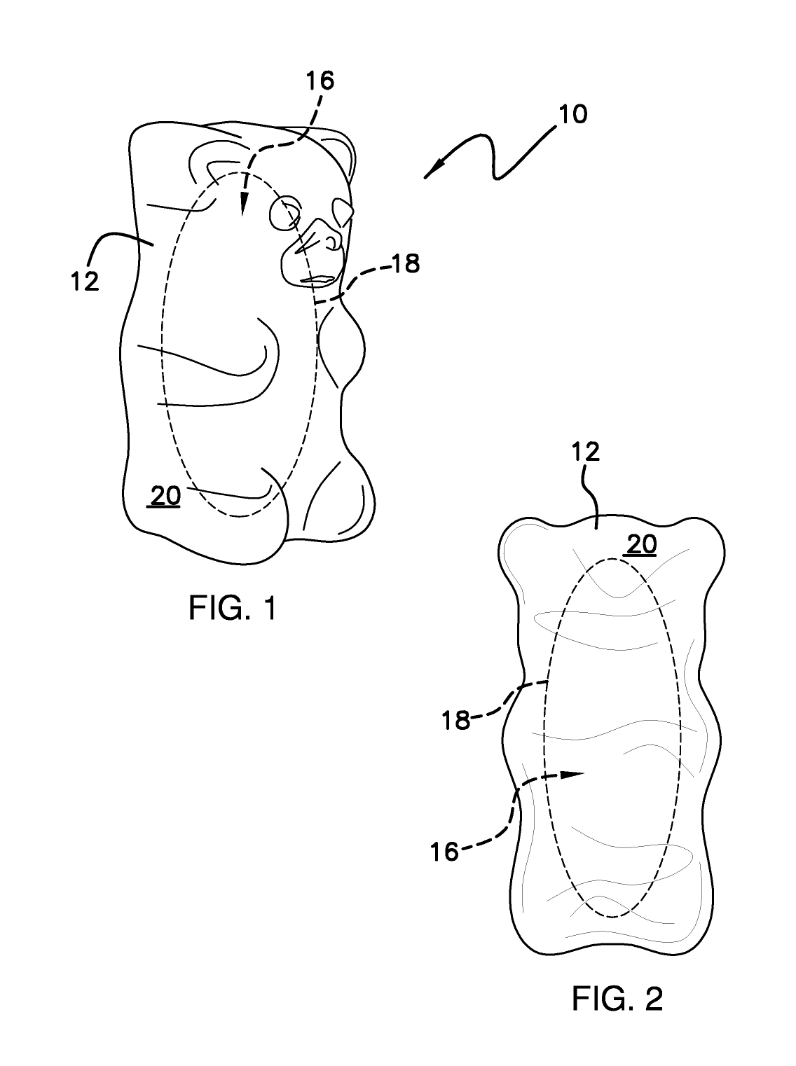

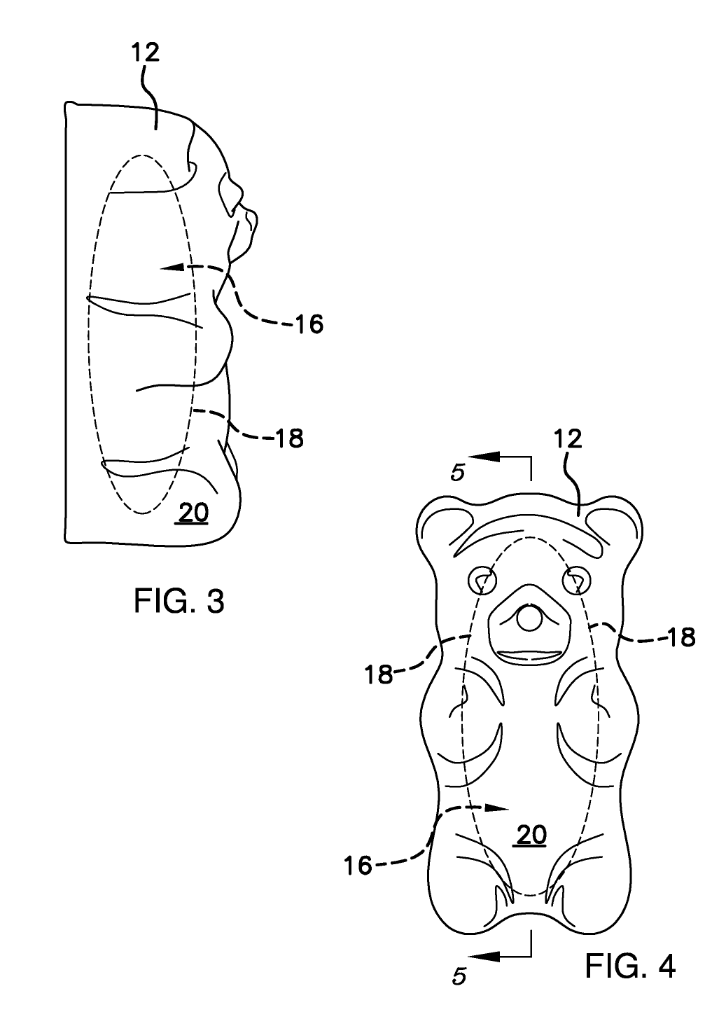

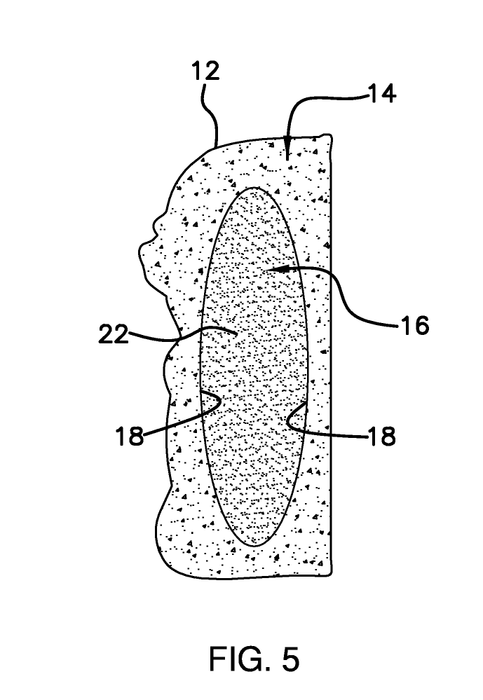

With reference now to the drawings, and in particular to FIGS. 1 through 5 thereof, a new medication delivery device embodying the principles and concepts of an embodiment of the disclosure and generally designated by the reference numeral 10 will be described.

As best illustrated in FIGS. 1 through 5, the children's medication delivery device 10 generally comprises a shell 12 comprising a gummy candy composition 14. The gummy candy composition 14 may be gelatinous in that gelatin may be incorporated into the composition 14. Alternatives to gelatin may be used as is conventionally known to provide similar texture and shape in a vegetarian form. The shell 12 defines an interior space 16 within the shell 12. The interior space 16 is defined by an interior wall 18 within the shell 12. The interior wall 18 is continuous within the shell 12 defining the interior space 14 as a prolate spheroid or substantially similar shape. The shell 12 has an outer surface 20 shaped to define an animal-like shape such as resembling a bear. A medication 22 is positioned in the interior space 14 of the shell 12 such that the medication 22 is enclosed by and contained in the shell 12. Thus, the medication 22 is not readily apparent to a child who may view the shell 12 as resembling candy to be enjoyed as opposed to medicine which might be associated with a bad taste or other characteristic children would dislike. The medication 22 is a liquid filling the interior space 16 fully. The medication 22 may be flavored either to correspond to flavoring of the composition 14 or to complement flavoring of the composition 14.

In use, the medication 22 is effectively disguised within the shell 12. The device 10 is provided to a person, most typically a young child who may be resistant to taking medications orally. The shell 12 is designed to mimic popular gummy candies such that the medication 22 within the shell 12 is readily ingested when offered to the young child as candy.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A children's medication delivery device comprising:
a shell, said shell comprising a gummy candy composition, said shell defining an interior space within said shell; and
a medication positioned in said interior space of said shell wherein said medication is enclosed by and contained in said shell.

2. The device of claim 1, further comprising said medication being a liquid.

3. The device of claim 2, further comprising said medication being flavored.

4. The device of claim 1, further comprising said interior space being defined by an interior wall within said shell, said interior wall defining said interior space as being prolate spheroid.

5. The device of claim 1, further comprising said shell having an outer surface shaped to define an animal-like shape.

6. The device of claim 5, further comprising said animal-like shape resembling a bear.

7. The device of claim 1, further comprising said gummy candy composition of said shell being gelatinous.

8. A children's medication delivery device comprising:
a shell, said shell comprising a gummy candy composition, said shell defining an interior space within said shell, said gummy candy composition of said shell being gelatinous, said interior space being defined by an interior wall within said shell, said interior wall defining said interior space as being prolate spheroid, said shell having an outer surface shaped to define an animal-like shape, said animal-like shape resembling a bear; and
a medication positioned in said interior space of said shell wherein said medication is enclosed by and contained in said shell, said medication being a liquid, said medication being flavored.
